(19) United States
(12) Patent Application Publication
(10) Pub. No.: US 2025/0025304 A1
(43) Pub. Date: Jan. 23, 2025

(54) CAP COMPONENT FOR AN ENDOPROSTHESIS, AND ENDOPROSTHESIS OR ARTIFICIAL JOINT COMPRISING SAID COMPONENT

(71) Applicant: MATHYS AG BETTLACH, Bettlach (CH)

(72) Inventors: Viola MAI, Fraubrunnen (CH); Victor VALDERRABANO, Hofstetten (CH)

(21) Appl. No.: 18/715,111

(22) PCT Filed: Jun. 15, 2022
(86) PCT No.: PCT/EP2022/066265

§ 371 Date: May 31, 2024

(30) Foreign Application Priority Data
Dec. 1, 2021 (DE) 10 2021 131 572.7
May 4, 2022 (DE) 10 2022 110 936.4

(51) Int. Cl. A61F 2/30; A61F 2/36

(52) U.S. Cl. CPC A61F 2/30734; A61F 2002/30069; A61F 2002/30131; A61F 2002/30245; A61F 2002/30327; A61F 2002/30632; A61F 2002/30878; A61F 2002/3611; A61F 2310/00407

(57) ABSTRACT

A cap component for an endoprosthesis includes a convex or flat sliding surface. The cap component is designed to enclose part or all of an associated bone joint part. The cap component and/or a first base material of the cap component have a modulus of elasticity that is about equal to bone material of the associated bone joint part. A facing surface of the cap component facing the associated bone joint part is at least partially coated with a first metal or with particles of the first metal.

### FIELD OF THE INVENTION

The invention relates to a cap component for an endoprosthesis (resurfacing of the joint part) and an endoprosthesis with such a cap component and an insert component (resurfacing of both joint parts).

### BACKGROUND

Typically, damaged joints in humans and animals are completely or partially replaced with endoprostheses. Using the example of a hip joint endoprosthesis, in particular a corresponding total endoprosthesis, it is common to replace both the acetabulum and the femoral head in each case as a whole. However, this typical procedure can be associated with a number of disadvantages, which can occur analogously with other joint types: Increased risk of bone fracture, particularly in the area of the artificial joint head, femoral neck impingement, malposition of the acetabulum, varus malposition of the femur, poor attachment of the endoprosthesis to the bone or poor osseointegration, necrosis, for example caused by the heat of the corresponding reamer used or by corresponding cementing, provoking of (pseudo) tumors and/or allergies, particularly due to metal particles or ions, typically made of cobalt or chromium, which are caused, for example, by abrasion in metal-on-metal endoprostheses. At least some of the aforementioned disadvantages are caused or favored by a relatively large bone resection associated with the corresponding implantation.

The document EP 2 872 072 B1 discloses a cup for a hip prosthesis which is capable of overcoming or reducing the risk of the above-mentioned disadvantage of metal particle release in the patient's body. Disadvantageously, the corresponding bone resection required for implant placement cannot be minimized even with the aid of the cup described.

Furthermore, document DE 10 2020 116 929 A1, which was published after the priority date of the present application, shows an endoprosthesis with an attachment region for direct or indirect attachment to or in a bone of a mammal and with a sliding surface which is arranged to delimit a joint gap of an at least partially artificial joint and to form a sliding pair with at least one artificial or natural joint component. In this context, it is essential that the attachment area is part of a prosthesis main body which at least partially comprises a polymer or a metal, and that the sliding surface is arranged on the prosthesis main body and comprises a ceramic material at least in some areas. However, even the aforementioned endoprosthesis is not able to sufficiently avoid or completely overcome the disadvantages described above.

### Problem and Solution

Accordingly, it is the object of the invention to provide a cap component for an endoprosthesis and also an endoprosthesis comprising such a cap component and an insert component, wherein in particular bone resection associated with the corresponding implantation is minimized.

The object is achieved with respect to the cap component for an endoprosthesis by the features of claim 1. The object is achieved with respect to the endoprosthesis comprising such a cap component and an insert component by the features of claim 15. The dependent claims contain advantageous developments.

### DETAILED DESCRIPTION OF THE INVENTION

According to a first aspect of the invention, a cap component for an endoprosthesis is created. The cap component is designed to enclose part or all of an associated bone joint part. Furthermore, the cap component and/or a base material of the cap component has a similar stiffness, in particular modulus of elasticity, to the bone material of the associated bone joint part, wherein the surface of the cap component facing the bone joint part is partially or completely coated with a first metal or with particles of a first metal. The cap component also has a convex or flat sliding surface.

It should also be noted that there is a particular advantage if the base material of the cap component also acts as a sliding material on the side facing the joint.

Advantageously, the aforementioned configuration allows the corresponding bone resection required for implantation of the cap component to be reduced to a minimum. In particular, the surface of the associated bone joint part can be renewed with minimal bone resection. Furthermore, the coating of metal or metal particles facilitates the growth of the cap component, which improves the corresponding osseointegration. In addition, the risk of necrosis, for example, is reduced, especially as the minimal bone resection during implantation means that hardly any heat is generated by a reamer. In addition, the use of cement can be completely dispensed with during implantation, which also prevents possible necrosis.

For the sake of completeness, it should also be mentioned that the cap component can also be used alone, i.e., in particular without interacting with other components, for example, artificial components such as artificial joint sockets for endoprostheses. Accordingly, it is possible for the cap component to interact directly with a human or animal bone joint part or for the sliding surface of the cap component to interact with a human or animal sliding surface.

According to a first preferred embodiment of the first aspect of the invention, the modulus of elasticity is less than 10 GPa, preferably less than 8 GPa, particularly preferably less than 5 GPa, very particularly preferably less than 1.5 GPa. Additionally or alternatively, the stiffness, in particular modulus of elasticity, of the sliding surface of the cap component is similar or identical to the stiffness, in particular modulus of elasticity, of the cap component and/or the base material of the cap component.

Advantageously, for example, the biomechanical properties of the corresponding endoprosthesis can be improved.

According to a second preferred embodiment of the first aspect of the invention, the base material of the cap component is softer than a second metal and/or a metal alloy, in particular of a base material of an insert component slidingly cooperating with the cap component. Additionally or alternatively, the second metal is preferably titanium, iron, chromium or cobalt. Further additionally or alternatively, the metal alloy preferably comprises at least one of the metals titanium, iron, chromium and cobalt.

Advantageously, the degree of hardness of the base material of the cap component can, for example, be further improved in the direction of optimal biomechanical properties of the corresponding endoprosthesis.

According to a further preferred embodiment of the first aspect of the invention, the first metal comprises titanium particles, the titanium particles having preferably been applied by means of a titanium plasma spray coating process.

As an alternative to the above-mentioned titanium plasma spray coating process, it can in principle be a thermal, thermal and pressurized, or non-thermal assisted coating process. In particular, it can be a pressing, sintering or vibration process or a press-fit process.

As a further alternative, the surface of the cap component facing the bone joint part can be partially or completely doped with the first metal or with particles of the second metal.

Advantageously, titanium or its particles have proven to be particularly suitable for improving the corresponding osseointegration.

As an alternative to titanium, it may be advantageous to use calcium, preferably a calcium compound, particularly preferably a calcium phosphate compound, very particularly preferably hydroxyapatite or bruschite.

According to a further preferred embodiment of the first aspect of the invention, the base material of the cap component comprises or consists of a plastic, preferably polyethylene, particularly preferably polyethylene stabilized with cross-links, very particularly preferably polyethylene mixed with vitamin E and stabilized with cross-links.

In particular, the aforementioned plastic may have a modulus of elasticity of at most 1 GPa. Furthermore, the plastic may alternatively be a non-metallic and non-ceramic plastic material. In particular, it may be a polymer, preferably highly cross-linked polyethylene or polyether ether ketone (PEEK) doped with vitamin E.

Advantageously, for example, an optimum with respect to the biomechanical properties of the corresponding endoprosthesis can be achieved. Furthermore, the base material or cap component is transparent to X-rays, which makes it particularly easy to monitor the associated bone joint part.

According to a further preferred embodiment of the first aspect of the invention, the cap component comprises at least one anti-rotation element, the at least one anti-rotation element preferably being arranged on the surface of the cap component facing the bone joint part.

Advantageously, a secure positioning of the cap component can be ensured in this way, which, for example, counteracts corresponding misalignments preventively.

According to a further preferred embodiment of the first aspect of the invention, the at least one anti-rotation element has the shape of a bar, bollard, thread, tooth, cross, star or anchoring pin, in particular peg. Additionally or alternatively, the at least one anti-rotation element is arranged circularly and/or at the corresponding pole in relation to the cap component.

Advantageously, the cap component can be positioned securely in a particularly efficient manner.

According to a further preferred embodiment of the first aspect of the invention, the cap component comprises at least one anti-translation element, the at least one anti-translation element preferably being arranged on the surface of the cap component facing the bone joint part.

Advantageously, a secure positioning of the cap component can be ensured in this way, which, for example, counteracts corresponding misalignments preventively.

According to a further preferred embodiment of the first aspect of the invention, the at least one anti-translation element has the form of a groove, preferably an annular groove, or at least one cavity, preferably several cavities, particularly preferably several cavities distributed uniformly over the corresponding surface, most preferably several golf-ball-like cavities.

Advantageously, the golf-ball-like structure has proven to be particularly suitable for secure positioning of the cap component, especially as the corresponding cavities can be produced in a particularly efficient manner.

According to a further preferred embodiment of the first aspect of the invention, the at least one anti-rotation element and/or the at least one anti-translation element are/is set up to absorb forces and/or moments immediately after the corresponding implantation and/or for the time after the corresponding growth of the bone material of the associated bone joint part.

The advantage of this is that malpositions can be efficiently prevented not only during implantation, but also later on. This means that the cap component is securely positioned for its entire service life.

According to a further preferred embodiment of the first aspect of the invention, the cap component comprises at least one recess, wherein the at least one recess is preferably designed to protect zones of critical blood supply during the corresponding preparation and/or implantation.

Advantageously, the aforementioned protection of zones with critical blood supply can be achieved in a particularly efficient or favorable manner.

According to a further preferred embodiment of the first aspect of the invention, the shape of the cap component is such that the shape deviates and/or tapers from the sphere of the surface facing away from the associated bone joint part, in particular below the corresponding equator of the cap component.

Preferably, the deviation and/or taper described above relates to the sliding surface of the cap component.

Advantageously, a corresponding impingement can be avoided or prevented, for example.

According to a further preferred embodiment of the first aspect of the invention, the shape has two different radii. Additionally or alternatively, the shape is based on an ellipse or a freely defined curve or line, whereby in particular a femoral head-neck offset of at least 0.5 mm, preferably of at least 2 mm, particularly preferably of at least 3 mm, very particularly preferably of at least 5 mm, is achieved.

According to a further preferred embodiment of the first aspect of the invention, the shape of the cap component is such that the shape deviates from the sphere of the surface facing the bone joint part, in particular below the corresponding equator of the cap component.

Preferably, the deviation described above does not concern the sliding surface of the cap component, but in particular the surface of the cap component, which is partially or completely coated with the first metal or with particles of the first metal.

Advantageously, the cap component can thus be fixed to the associated bone joint part in a particularly secure and efficient manner, even if no cementing is performed.

According to a second aspect of the invention, an endoprosthesis is provided comprising an insert component for a first bone, in particular a first bone joint part, wherein the insert component has a concave or flat sliding surface, and a cap component according to one of the embodiments of the first aspect of the invention for a second bone, in particular a second bone joint part, wherein the sliding surface of the insert component is in sliding contact with the sliding surface of the cap component.

In addition to the numerous advantages of the insert component and the cap component, the sliding system is advantageously not a metal-on-metal system, which means that no metal particles enter the corresponding organism, which prevents (pseudo) tumors or allergies, for example.

It should be mentioned that the insert component can be, in particular, a joint socket, for example, a hip joint socket.

According to a first preferred embodiment of the second aspect of the invention, the insert component is designed to be inserted into an associated bone joint part, in particular the first bone joint part, or to partially or completely replace it. Additionally or alternatively, the surface of the insert component facing the bone joint part, in particular the first bone joint part, is partially or completely coated with a first metal or with particles of a first metal. Furthermore, additionally or alternatively, a base material of the insert component comprises a second metal or a metal alloy and/or a metal oxide or a metal oxide mixture.

It can be particularly advantageous if the modulus of elasticity of the base material of the insert component and the modulus of elasticity of the base material of the cap component differ by at least a factor of 50, preferably by at least a factor of 70, particularly preferably by at least a factor of 100, very particularly preferably by at least a factor of 200. In this context, in particular the modulus of elasticity of the insert component is greater than that of the cap component.

Advantageously, the aforementioned configuration allows the corresponding bone resection required for implantation of the insert component to be reduced to a minimum. In particular, the surface of the associated bone joint part can be renewed with minimal bone resection. Furthermore, the coating of metal or metal particles facilitates the growth of the insert component, which improves the corresponding osseointegration. In addition, the risk of necrosis, for example, is reduced, especially as the minimal bone resection during implantation means that hardly any heat is generated by a reamer. In addition, the use of cement can be completely dispensed with during implantation, which also preventatively counteracts possible necrosis.

According to a second preferred embodiment of the second aspect of the invention, the metal oxide is or comprises yttrium oxide, zirconium oxide or aluminum oxide. Additionally or alternatively, the metal oxide mixture comprises at least one of the metal oxides yttrium oxide, zirconium oxide or aluminum oxide or is a mixture of yttrium oxide, stabilized zirconium oxide and aluminum oxide. Additionally or alternatively, the base material of the cap component is softer than the second metal and/or the metal alloy of the insert component.

Advantageously, the aforementioned oxides have proven to be particularly suitable for renewing the surface of the associated bone joint part with minimal bone resection.

According to a further preferred embodiment of the second aspect of the invention, the first metal of the insert component comprises titanium particles, the titanium particles having preferably been applied by means of a titanium plasma spray coating process.

Advantageously, titanium or its particles have proven to be particularly suitable for improving the corresponding osseointegration.

According to a further preferred embodiment of the second aspect of the invention, the insert component comprises at least one rotation and/or anti-translation element, the at least one rotation and/or anti-translation element preferably being arranged on the surface of the insert component facing the bone joint part.

Advantageously, a secure positioning of the insert component can be ensured in this way, which, for example, counteracts corresponding misalignments preventively.

According to a further preferred embodiment of the second aspect of the invention, the at least one rotation and/or anti-translation element has the form of at least one tooth, preferably a toothed macrostructure, or at least one cavity, preferably several cavities, particularly preferably several cavities distributed uniformly over the corresponding surface, most preferably several golf-ball-like cavities.

Advantageously, the golf-ball-like structure has proven to be particularly suitable for secure positioning of the insert component, especially as the corresponding cavities can be produced in a particularly efficient manner.

According to a further preferred embodiment of the second aspect of the invention, the first bone is a pelvis and the first bone joint part is an acetabulum. In addition, the second bone is a femur and the second bone joint part is a caput femoris.

Advantageously, a hip joint can be treated, although it is not completely replaced, but rather the corresponding surfaces of the bone joint parts are renewed or corrected. This minimizes the corresponding bone resection during implantation.

It is particularly advantageous if the corresponding sliding surfaces are preferably replaced or renewed.

The embodiments described below show that the endoprosthesis according to the invention comprising an insert component according to the invention and a cap component according to the invention can advantageously be used particularly flexibly.

According to a further preferred embodiment of the second aspect of the invention, the first bone is a tibia. In addition, the second bone is a talus.

According to a further preferred embodiment of the second aspect of the invention, the first bone is a tibia. In addition, the second bone is a femur.

According to a further preferred embodiment of the second aspect of the invention, the first bone is a phalanx. In addition, the second bone is a metatarsal bone.

According to a further preferred embodiment of the second aspect of the invention, the first bone is a glenoid. In addition, the second bone is a humerus.

According to a further preferred embodiment of the second aspect of the invention, the first bone is a radius. In addition, the second bone is a scaphoid bone.

According to a further preferred embodiment of the second aspect of the invention, the first bone and the first bone joint part are part of a human skeleton. In addition, the second bone and the second bone joint part are part of a human skeleton.

According to a further preferred embodiment of the second aspect of the invention, the first bone and the first bone joint part are part of an animal skeleton. In addition, the second bone and the second bone joint part are part of an animal skeleton.

### DESCRIPTION OF PREFERRED EMBODIMENTS

In the following, a detailed exemplary description of some embodiments of the invention is given with reference to the figures of the drawing. In the figures:

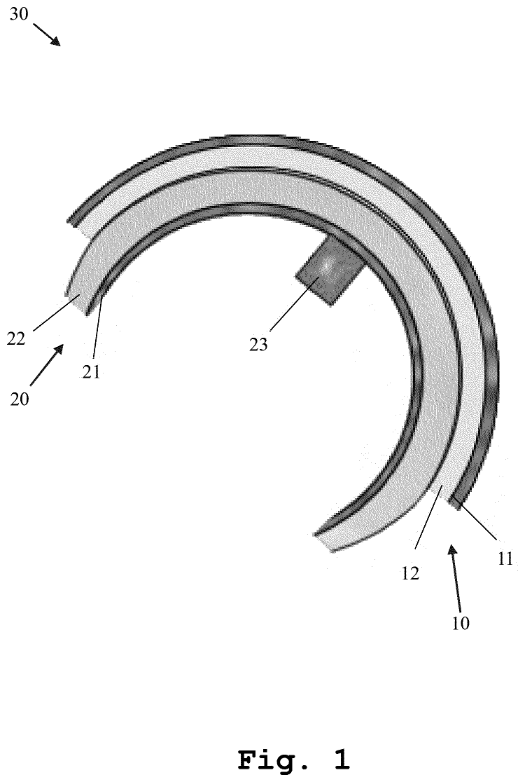

FIG. 1 shows an exemplary embodiment of an endoprosthesis according to the invention;

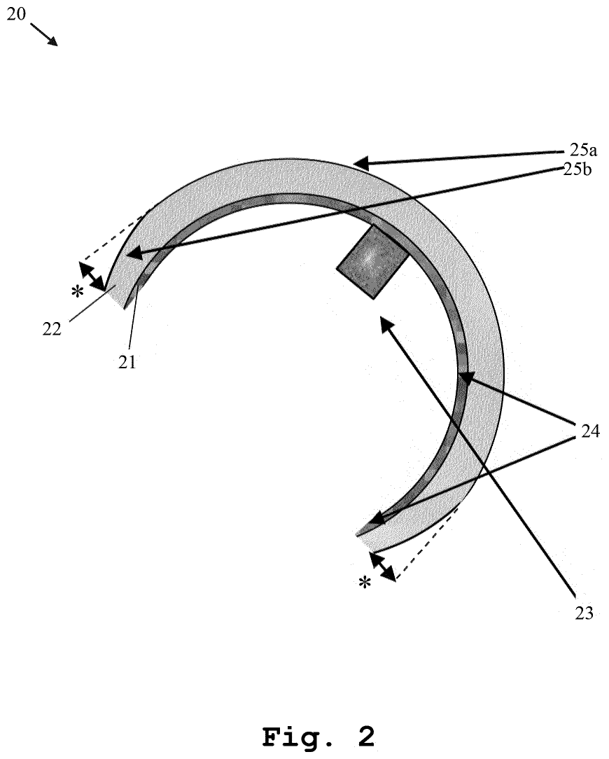

FIG. 2 shows the cap component of the endoprosthesis according to FIG. 1 alone;

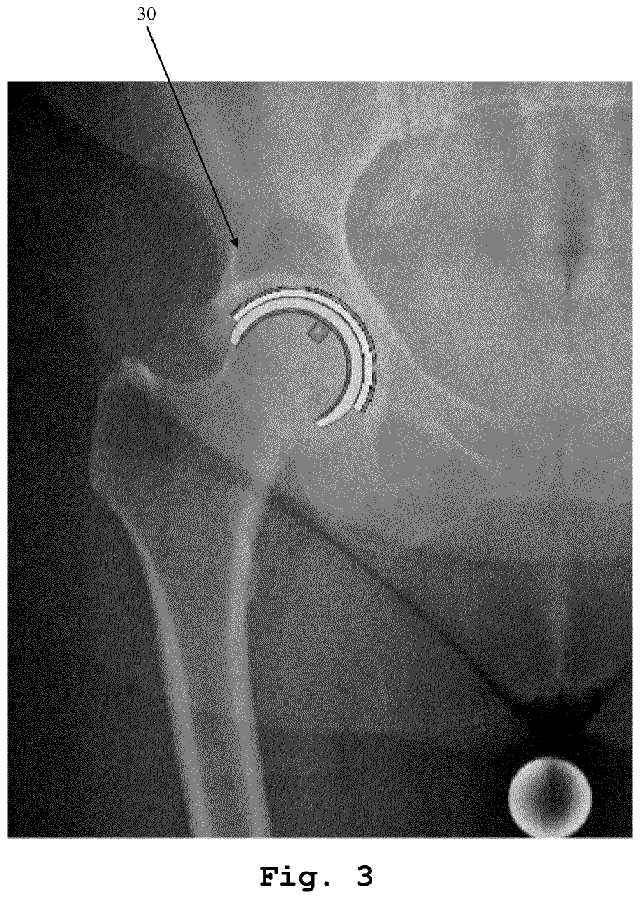

FIG. 3 shows an exemplary use of the endoprosthesis according to FIG. 1 as a hip endoprosthesis;

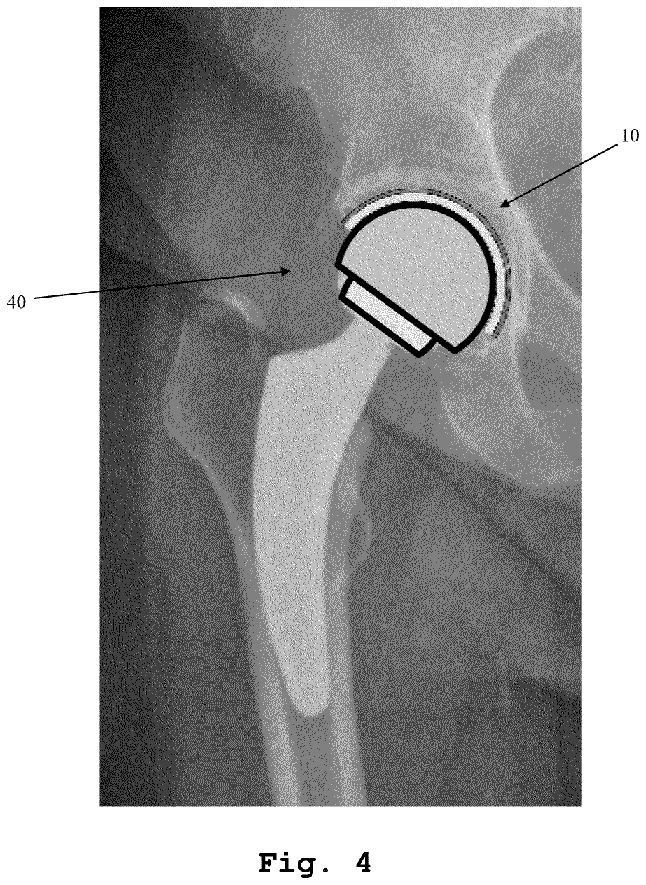

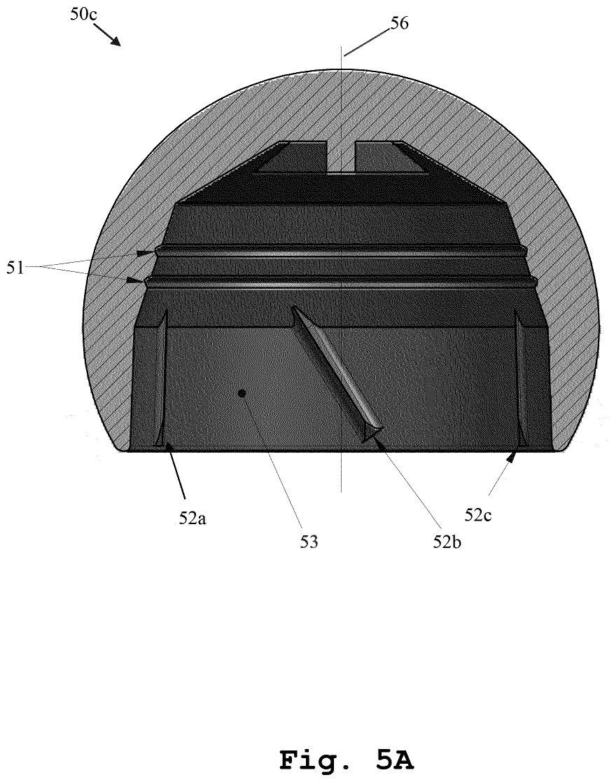

FIG. 4 shows a possible further use of at least part of the hip endoprosthesis according to FIG. 3 in the case of a follow-up operation;

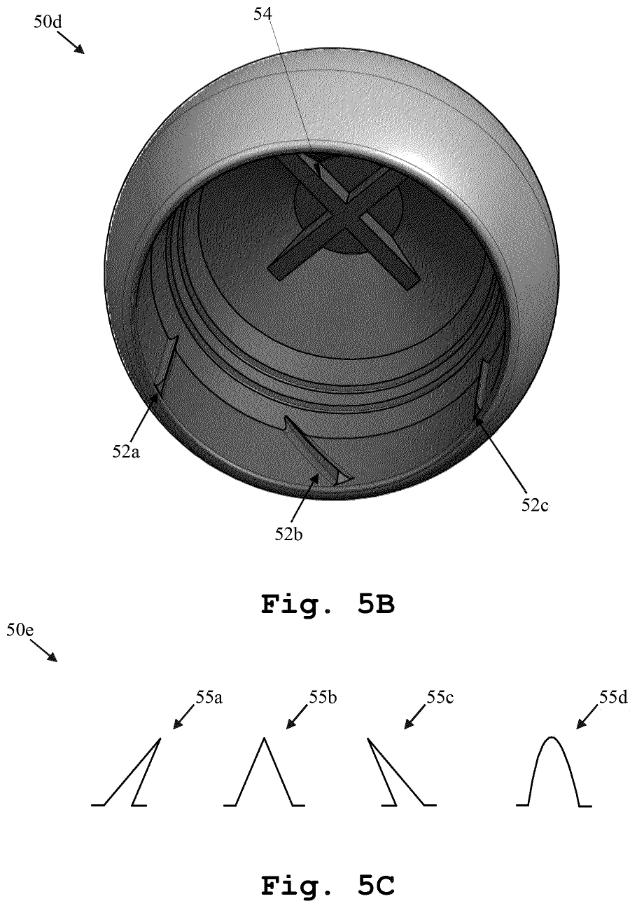

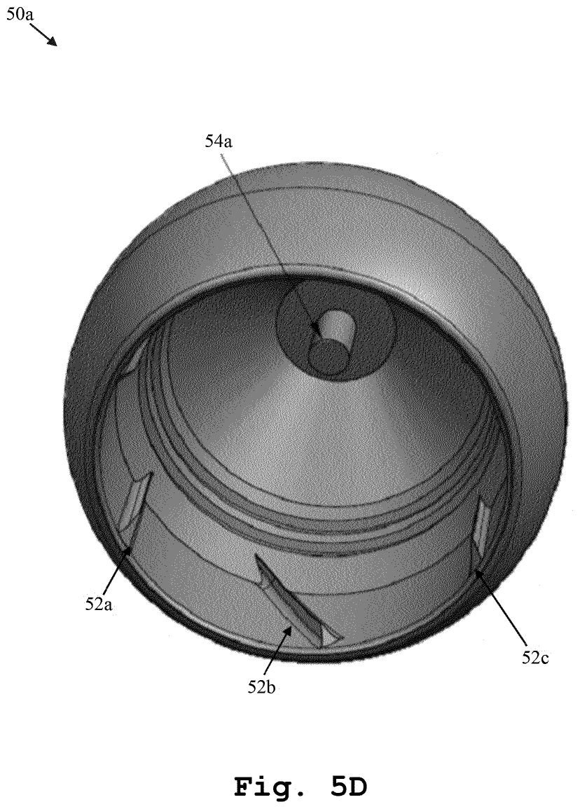

FIG. 5A shows a detailed sectional drawing of an exemplary embodiment of a cap component according to the invention;

FIG. 5B shows a further view of the embodiment according to FIG. 5A;

FIG. 5C shows exemplary rib shapes of the ribs comprised by the embodiment according to FIG. 5A;

FIG. 5D shows a view of a further exemplary embodiment of a cap component according to the invention;

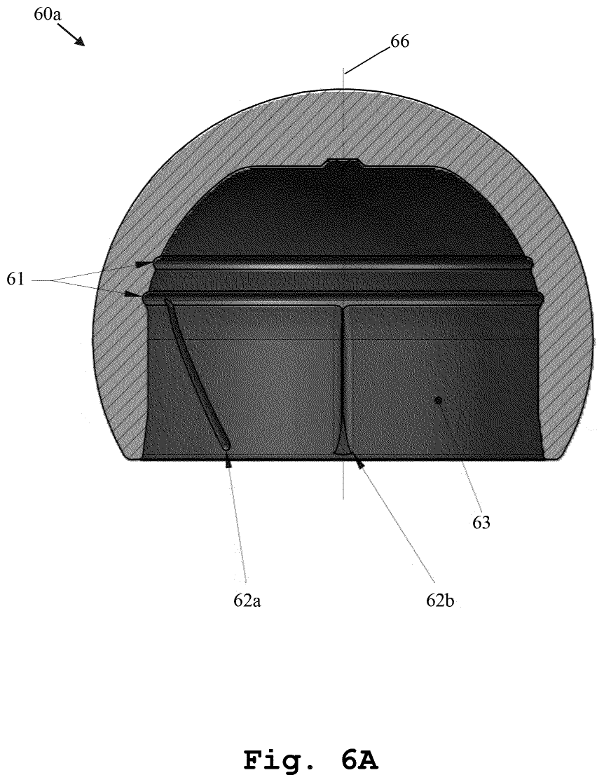

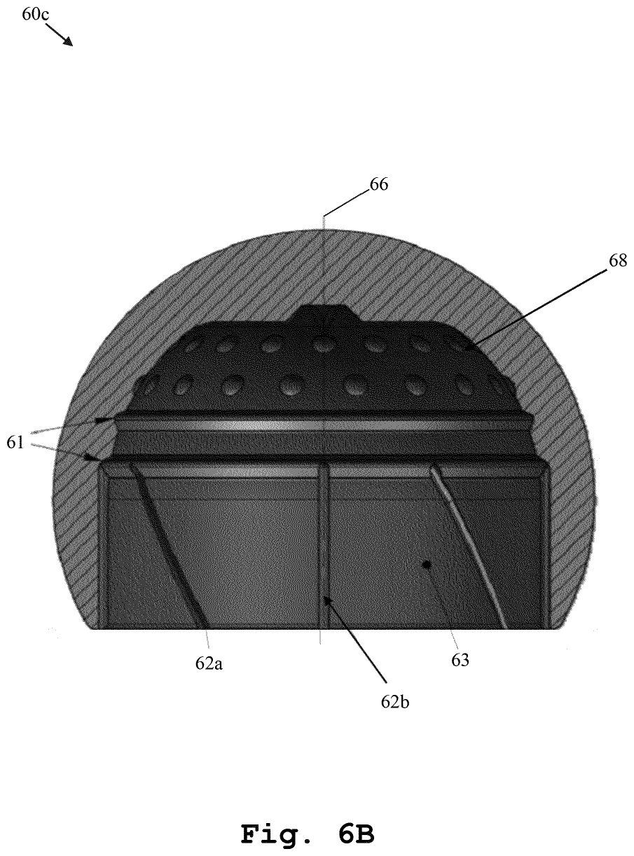

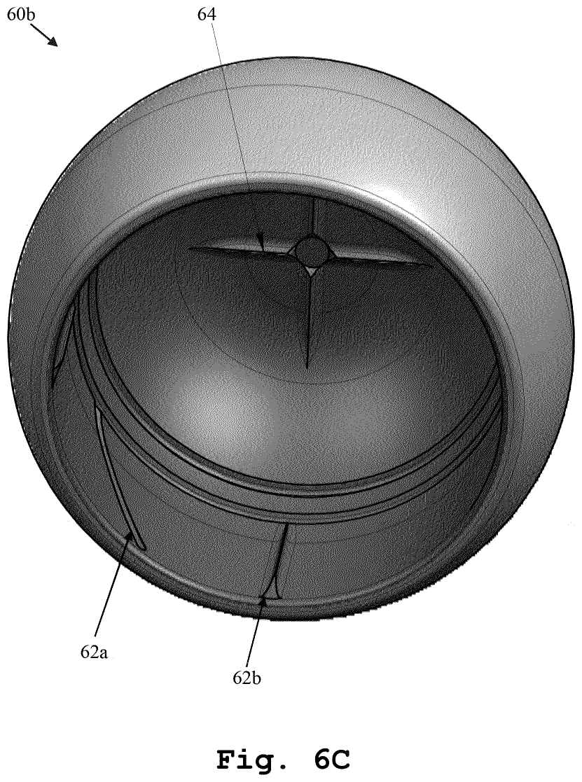

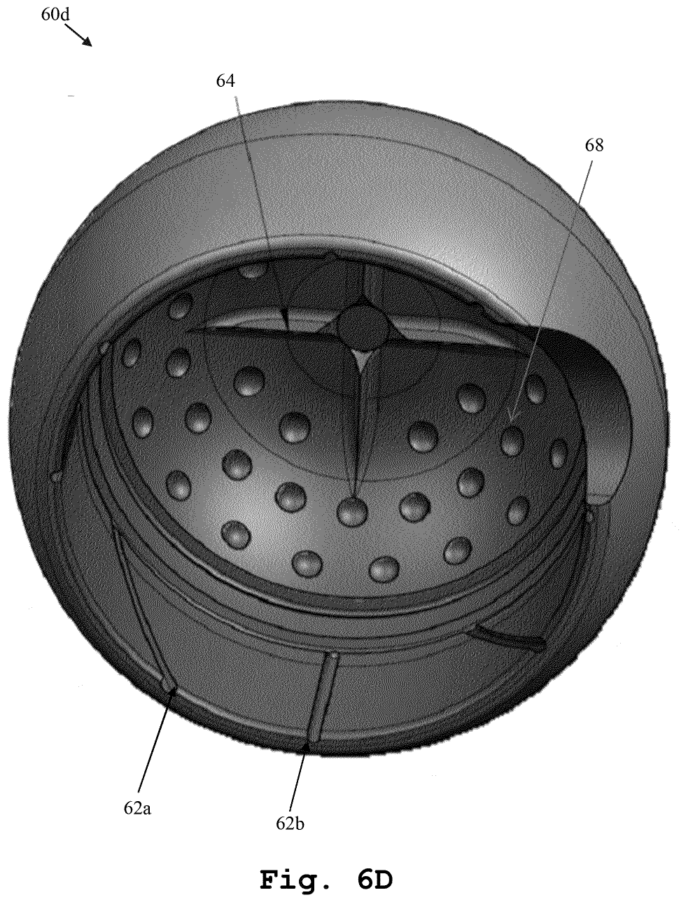

FIG. 6A shows a detailed sectional drawing of a further exemplary embodiment of a cap component according to the invention;

FIG. 6B shows a detailed sectional drawing of a further exemplary embodiment of a cap component according to the invention;

FIG. 6C shows a view of a three-dimensional illustration of the embodiment according to FIG. 6A;

FIG. 6D shows a view of a three-dimensional illustration of the embodiment according to FIG. 6B;

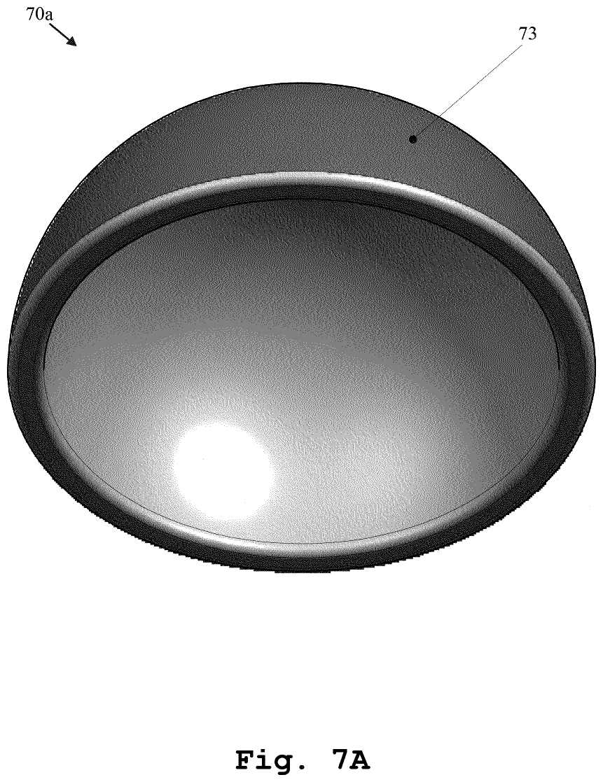

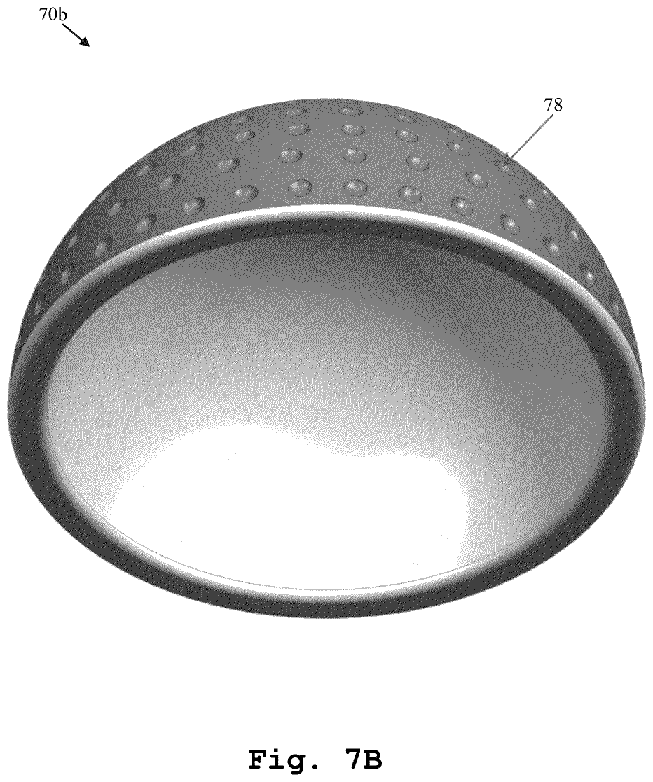

FIG. 7A shows a three-dimensional illustration of an exemplary embodiment of an insert component according to the invention;

FIG. 7B shows a three-dimensional illustration of a further exemplary embodiment of an insert component according to the invention;

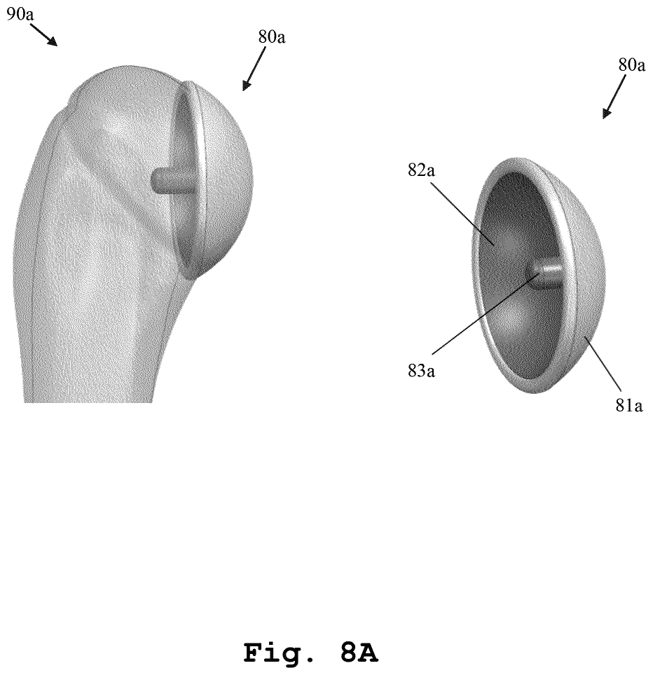

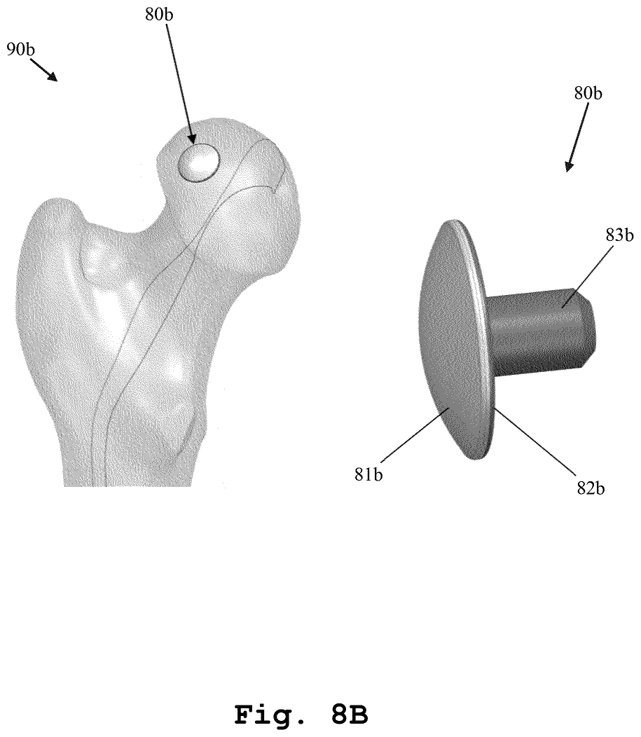

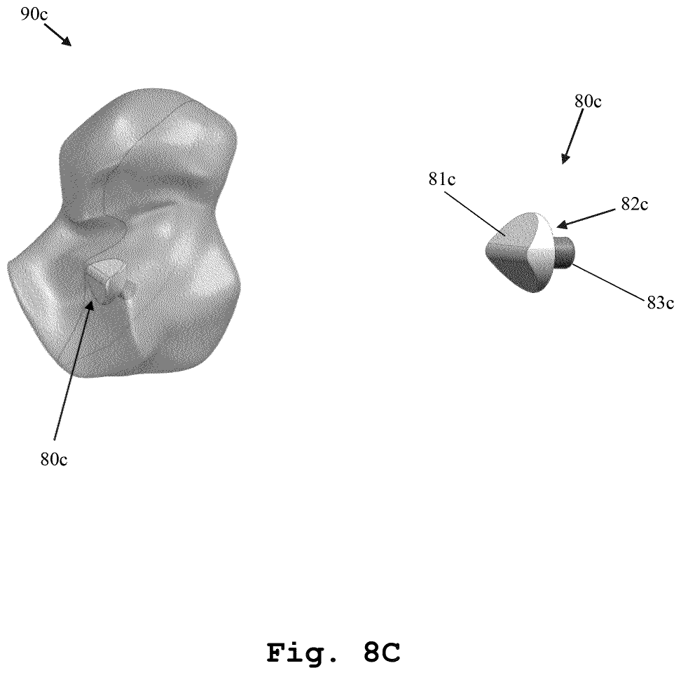

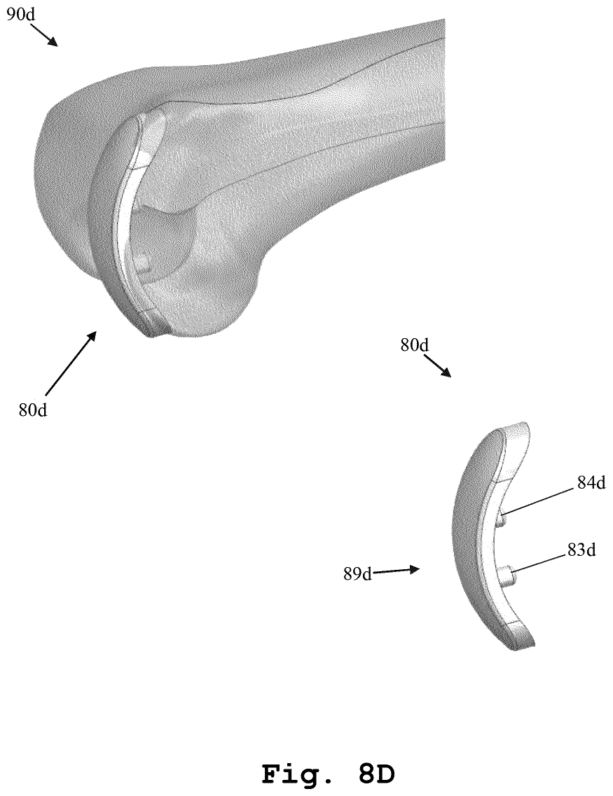

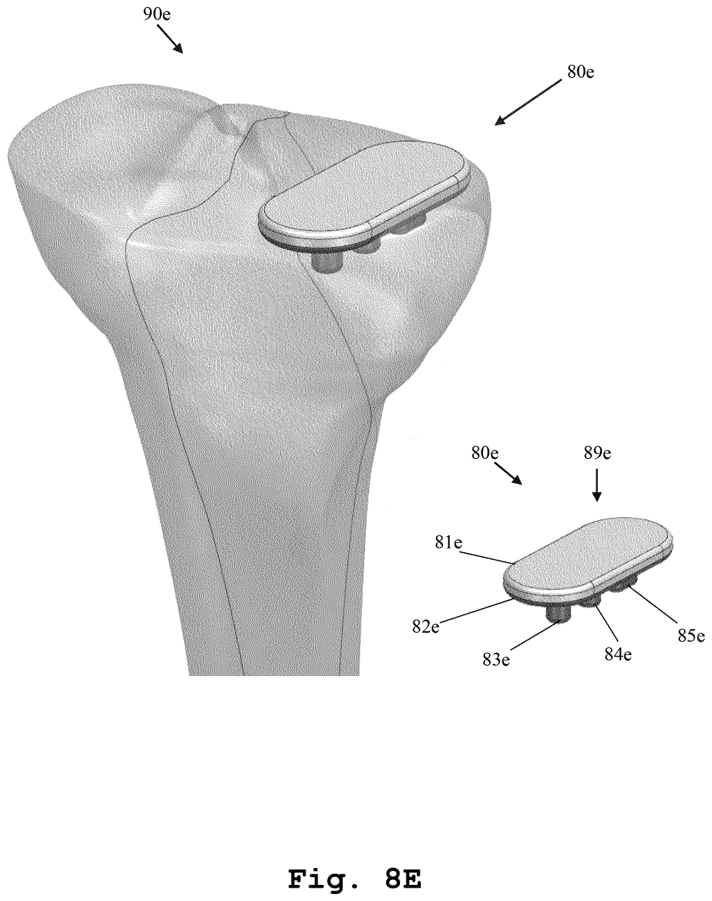

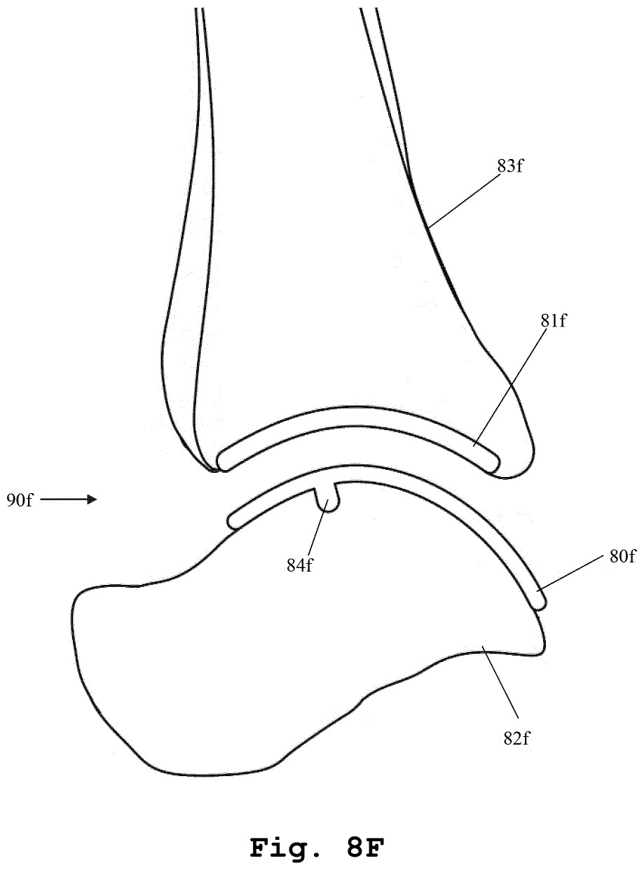

FIG. 8A shows an illustration of a further exemplary embodiment of a cap component according to the invention, in particular in conjunction with a humerus;

FIG. 8B shows an illustration of a further exemplary embodiment of a cap component according to the invention, in particular in conjunction with a femoral head;

FIG. 8C shows an illustration of a further exemplary embodiment of a cap component according to the invention, in particular in conjunction with a talus;

FIG. 8D shows an illustration of a further exemplary embodiment of a cap component according to the invention, in particular in conjunction with a femoral condyle;

FIG. 8E shows an illustration of a further exemplary embodiment of a cap component according to the invention, in particular in conjunction with a tibia; and

FIG. 8F shows an illustration of a further exemplary embodiment of a cap component according to the invention, in particular in conjunction with a tibia-talus joint.

FIG. 1 illustrates an exemplary embodiment of an endoprosthesis 30 according to the invention with an exemplary embodiment of an insert component 10 according to the invention for a first bone, in particular a first bone joint part, and an exemplary embodiment of a cap component 20 according to the invention for a second bone, in particular a second bone joint part, wherein the sliding surface of the insert component 10 is in sliding contact with the sliding surface of the cap component 20.

The surface 11 of the insert component 10 facing the bone joint part is partially or completely coated with a first metal or with particles of a first metal. In addition, the insert component 10 has a sliding surface that is convex or flat.

The insert component 10 is also designed to be inserted into an associated bone joint part or to partially or completely replace it. A base material 12 of the insert component 10 basically comprises a second metal or a metal alloy and/or a metal oxide or a metal oxide mixture.

With regard to the above-mentioned metal oxide, it should be noted that it may contain or may be yttrium oxide, zirconium oxide or aluminum oxide. Additionally or alternatively, the metal oxide mixture may comprise at least one of the metal oxides yttrium oxide, zirconium oxide or aluminum oxide or may be a mixture of yttrium oxide, stabilized zirconium oxide and aluminum oxide.

The insert component 10 of the endoprosthesis 30 has, by way of example, a base material 12 which comprises a mixture of yttrium-stabilized zirconium oxide and aluminum oxide.

Furthermore, it is particularly advantageous if the first metal comprises titanium particles, the titanium particles preferably having been applied using a titanium plasma spray coating process or another thermal process. For example, the insert component 10 has such a coating 11. In particular, this is a TiCP coating.

It can also be particularly advantageous if the insert component 10 comprises at least one rotation and/or anti-translation element, the at least one rotation and/or anti-translation element preferably being arranged on the surface 11 of the insert component 10 facing the bone joint part.

Even if the exemplary embodiment 10 of the insert component according to the invention according to FIG. 1 does not explicitly show such a securing element, it should be noted that the at least one rotation and/or anti-translation element has the form of at least one tooth, preferably a toothed macrostructure, or at least one cavity, preferably several cavities, particularly preferably several cavities distributed uniformly over the corresponding surface, very particularly preferably several golf-ball-like cavities.

With regard to the cap component 20 for the endoprosthesis 30, it should be mentioned that the cap component 20 is designed to partially or completely enclose an associated bone joint part. Accordingly, the cap component 20 according to the invention serves in particular for surface renewal or resurfacing with respect to the associated bone joint part. The associated bone joint part itself can typically be preserved.

In addition, the surface 21 of the cap component 20 facing the bone joint part is partially or completely coated with a first metal or with particles of a first metal. Furthermore, a base material 22 of the cap component 20 is generally softer than a second metal and/or a metal alloy, in particular than the second metal and/or the metal alloy of the insert component 10. The cap component 20 additionally has a convex or flat sliding surface.

Moreover, it can be particularly advantageous if the cap component 20 and/or the base material 22 of the cap component 20 has a similar stiffness, in particular modulus of elasticity, to the bone material of the associated bone joint part. It should also be noted that the second metal can be titanium, iron, chromium or cobalt.

Additionally or alternatively, the metal alloy may comprise at least one of the metals titanium, iron, chromium and cobalt. In addition, it may be particularly advantageous if the first metal comprises titanium particles, the titanium particles preferably having been anchored by high- or low-temperature processes. For example, the cap component 20 has such a coating 21 of pure titanium. In particular, this can also be a TiCP coating.

A further particular advantage arises if the base material 22 of the cap component 20 comprises or consists of a plastic, preferably polyethylene, particularly preferably polyethylene stabilized with cross-links, very particularly preferably polyethylene mixed with vitamin E and stabilized with cross-links.

By way of example, the base material 22 of the cap component 20 comprises polyethylene blended with vitamin E. Furthermore, according to FIG. 1, it can be seen that the cap component 20 has at least one anti-rotation element 23, wherein the at least one anti-rotation element 23 is preferably arranged on the surface 21 of the cap component 20 facing the bone joint part.

It can also be particularly advantageous if the at least one anti-rotation element 23 has the form of a bar, bollard, thread, tooth, cross, star or anchoring pin, in particular a peg. Additionally or alternatively, there is a particular advantage if the at least one anti-rotation element 23 is arranged circularly and/or at the corresponding pole with respect to the cap component 20.

In the case of the anti-rotation element 23 according to FIG. 1, for example, it is a peg, in particular made of polyethylene or of the base material 22 of the cap component 20, with a cross base. In addition, the cap component 20 can have at least one anti-translation element, wherein the at least one anti-translation element is preferably arranged on the surface 21 of the cap component 20 facing the bone joint part.

Even if the exemplary embodiment 20 of the cap component according to the invention according to FIG. 1 does not explicitly show such a securing element, it should be noted that the at least one anti-translation element has the form of a groove, preferably an annular groove, or at least one cavity, preferably several cavities, particularly preferably several cavities distributed uniformly over the corresponding surface, very particularly preferably several golf-ball-like cavities.

Furthermore, a particular advantage can arise if the at least one anti-rotation element 23 and/or the at least one anti-translation element are/is set up to absorb forces and/or moments immediately after the corresponding implantation and/or for the time after the corresponding growth of the bone material of the associated bone joint part.

Furthermore, it should be mentioned that the cap component 20 may comprise at least one recess, wherein the at least one recess would preferably be designed to protect zones with critical blood supply during the corresponding preparation and/or implantation.

It can also be seen from FIG. 1 that the shape of the cap component 20 is such that the shape deviates and/or tapers from the sphere of the surface facing away from the associated bone joint part, in particular below the corresponding equator of the cap component 20.

In this context, it should also be noted that the aforementioned shape has two or more different radii. It should also be mentioned that the shape can additionally or alternatively be based on an ellipse or a freely defined curve.

With regard to the two or more radii, it should also be mentioned that it can be particularly advantageous if three radii are present, with one of the three radii in particular being a transition radius with respect to the other two radii.

Furthermore, a particular advantage can arise if the aforementioned shape of the cap component 20 is such that the shape deviates from the sphere of the surface 21 facing the bone joint part, in particular below the corresponding equator of the cap component 20. With regard to the two different radii already mentioned above, which the shape has, it should be noted that these are additionally emphasized by FIG. 2, which shows the cap component 20 alone.

According to FIG. 2, the surface facing away from the associated bone joint part has two outer radii 25a, 25b, can additionally or alternatively be based on more than two radii, ellipsoids or a freely defined curve or line, while the surface 21 facing the bone joint part has an inner arc 24, which advantageously merges into a cylindrical or slightly conical portion at the exit. In particular in the event that the endoprosthesis 30 is used as a hip endoprosthesis, which is shown by way of example in FIG. 3, the two asterisks (*) in FIG. 2 additionally illustrate that the two outer radii 25a, 25b are designed to achieve a femoral head-neck offset of at least 0.5 mm, preferably of at least 2 mm, particularly preferably of at least mm, very particularly preferably of at least 5 mm.

In the case of FIG. 3, it should be mentioned for the sake of completeness that the first bone mentioned in the context of the endoprosthesis 30 is a pelvis and the first bone joint part is an acetabulum, and that the second bone also mentioned in the context of the endoprosthesis 30 is a femur and the second bone joint part is a caput femoris.

FIG. 4 also illustrates a further advantage of the endoprosthesis 30: Particularly in the event that the above-mentioned surface replacement using the cap component 20 is not sufficient in the long term, the femur or the femoral head can be replaced by a conventional implant 40 in a corresponding follow-up operation, while at least a part, in particular the insert component 10, of the endoprosthesis 30 typically remains unchanged.

Since at least part of the endoprosthesis 30—even in the case of a follow-up operation-does not have to be adapted or can continue to be used substantially unchanged, a corresponding follow-up operation is not only less complicated, but also shorter in duration.

A detailed sectional drawing 50c of an exemplary embodiment of a cap component according to the invention is shown in FIG. 5A, which for example comprises two grooves 51, which in particular run parallel to one another. Preferably, these two grooves 51 serve as an anti-translation means or anti-translation elements and are located in particular in the tapered part of the cap component. The course of the grooves is typically perpendicular or substantially perpendicular to an axis 56 or also not parallel at an angle to each other, wherein the axis 56 runs in particular centrally or substantially centrally through the pole of the cap component.

Furthermore, according to FIG. 5A, it can be seen that the surface facing the bone joint part or the inner surface of the cap component is provided with a TiCP coating 53 or osteoconductive coating. In addition, the cap component comprises ribs 52a, 52b, 52c, which preferably serve as an anti-rotation means or anti-rotation elements and are arranged on the inner surface of the cap component. While the ribs 52a and 52c each run parallel or substantially parallel to the axis 56, in the exemplary embodiment the rib 52b is arranged tilted with respect to the axis 56.

It should be noted that the ribs 52a, 52b, 52c are shown together in the illustration 50c, in particular for the sake of compactness, although in practice either the ribs 52a and 52c are typically implemented together or one of the two aforementioned ribs alone or the rib 52b alone. In other words, in practice, elements are generally arranged on the inner surface of the cap component such that they are either tilted or parallel or substantially parallel to the axis 56.

Furthermore, it can be seen from FIG. 5A that all ribs 52a, 52b, 52c have a triangular cross-section. In particular, the rib 52b has the cross-section of an equilateral triangle, while the ribs 52a and 52c each have the cross-section of a right-angled triangle.

In the case of the aforementioned right-angled triangle, the shorter cathetus is preferably arranged on the inner surface of the cap component. In addition, the two ribs 52a and 52c are oriented in such a way that their cross-sections cannot be brought into alignment if one of the two ribs 52a and 52c is moved on the inner surface of the cap component.

Furthermore, FIG. 5B shows a further view 50d of the illustration according to FIG. 5A. The aforementioned view 50d shows in particular the ribs 52a, 52b, 52c already mentioned above as well as a rib cross 54, which is arranged on the inner surface at the pole of the cap component. Preferably, this rib cross 54 serves to prevent the rotation of the cap component or as an anti-rotation element. It should be noted that the rib cross 54 is arranged in particular perpendicularly or substantially perpendicularly to the axis 56, which is not explicitly shown in the illustration 50d. In addition, the axis 56 penetrates the center of the rib cross 54 in particular.

It should also be noted that the ribs 52a, 52b, 52c can also have other shapes or cross-sections. As an example, FIG. 5C shows further possible rib shapes 50e and their cross-sections 55a, 55b, 55c, 55d. According to the cross-sections 55a and 55c, for example, inclined triangles that are inclined in different directions can be considered as suitable cross-sections. Furthermore, an isosceles triangle 55b is also suitable as a cross-section for a rib as well as a parabolic or sinusoidal cross-section.

Furthermore, FIG. 5D shows a view of a further exemplary embodiment 50a of a cap component according to the invention.

It can be seen that this embodiment 50a substantially corresponds to that shown in FIG. 5C, wherein the rib cross 54 has been replaced by a pin 54a.

In this context, the above illustrations, in particular the different rib shapes according to FIG. 5C, apply analogously.

FIG. 6A shows a detailed sectional drawing 60a of a further exemplary embodiment of a cap component according to the invention. It should be noted that FIG. 5A, FIG. 5B, FIG. 5C and FIG. 5D already explained can apply analogously to this embodiment.

It should also be pointed out that the above remarks regarding the axis 56 and the grooves 51 apply analogously both to the axis 66 shown in FIG. 6A and to the two grooves 61. The grooves 61 and 51 are typically annular grooves.

In addition, FIG. 6A shows a groove 62b which is arranged parallel or substantially parallel to the axis 66 on the inner surface of the cap component. The rib shapes according to FIG. 5C can apply to this groove 62b in an analogous and, in particular, inverted manner. In this context, “inverted” means that the rib is designed as an inward-facing element in the form of a groove.

Furthermore, the surface facing the bone joint part or the inner surface of the cap component has a groove 62a, which is arranged tilted with respect to the axis 66. Preferably, this groove 62a serves as an anti-rotation and/or anti-translation means or anti-rotation and/or anti-translation element. Furthermore, according to FIG. 6A, it can be seen that the surface facing the bone joint part or the inner surface of the cap component is provided with a coating 63, in particular a TiCP coating. In addition, the groove 62a—analogous to the groove 62b—can also be a rib according to FIG. 5C, which has been designed accordingly in an inverted form.

Furthermore, FIG. 6B shows a detailed sectional drawing of a further exemplary embodiment 60c of a cap component according to the invention, which is substantially the same as that according to FIG. 6A, wherein the embodiment 60c additionally comprises a golf-ball-like structure 68 which is arranged on the inner surface of the cap component.

Advantageously, this golf-ball-like structure 68 secures the cap component in particular against corresponding rotation and/or translation, preferably rotation, and can be replaced in its geometric configuration by other inwardly directed shapes.

Furthermore, it is particularly advantageous if the golf ball-like structure 68 is arranged in such a way that it begins above the grooves 61 in the direction of the pole of the cap component and, in particular, extends to the pole.

FIG. 6C illustrates a view 60b of a three-dimensional illustration of the embodiment according to FIG. 6A, which additionally shows a rib cross 64, for which the above explanations regarding the rib cross 54 can apply analogously. Preferably, this rib cross 64 serves to prevent the rotation of the cap component or as an anti-rotation element. It should be noted that the rib cross 64 is arranged in particular perpendicularly or substantially perpendicularly to the axis 66, which is not explicitly shown in the illustration 60b. In addition, the axis 66 penetrates the center of the rib cross 64 in particular.

Similarly, FIG. 6D shows a view 60d of a three-dimensional illustration of the embodiment according to FIG. 6B.

For the sake of completeness, it should be noted that the rib crosses 54 and 64 differ substantially in that the leg surfaces of the rib cross 54 facing the bone joint part are each flat, while the leg surfaces of the rib cross 64 are each pointed towards the bone joint part.

FIG. 7A further shows a three-dimensional illustration 70a of an exemplary embodiment of an insert component according to the invention. According to FIG. 7A, it can be seen that the surface of the insert component facing the bone joint part is provided with a coating 73, preferably of pure titanium or an osteoconductive layer.

Furthermore, FIG. 7B depicts a three-dimensional illustration 70b of a further exemplary embodiment of an insert component according to the invention. This substantially corresponds to that shown in FIG. 7A, wherein the surface facing outwards, towards the bone, has golf-ball-like indentations 78 or a golf-ball-like structure. This serves to prevent rotation and/or translation, in particular rotation, and can be replaced in its geometric form by other inward-facing shapes.

Lastly, different embodiments of a cap component according to the invention are presented below in the context of correspondingly different bones or bone joint parts, in particular in order to demonstrate the high flexibility of the invention.

FIG. 8A shows an embodiment 80a of a cap component according to the invention, which is used in conjunction with a humerus 90a.

The embodiment 80b of the cap component according to the invention as shown in FIG. 8B, on the other hand, is used in conjunction with a femoral head 90b.

Furthermore, FIG. 8C illustrates an embodiment 80c of the cap component according to the invention, which is used in conjunction with a talus 90c. Here, the cap component 80c is particularly suitable for achieving partial resurfacing, preferably at the corresponding edge area.

For the sake of completeness, it should be mentioned that a cap component according to the invention is generally suitable for partial joint resurfacing.

The aforementioned embodiments 80a, 80b, 80c of the cap component according to the invention in particular each have a base material 81a, 81b, 81c made of non-metallic and non-ceramic plastic material, preferably with a modulus of elasticity of at most 5 GPa. The plastic material can be a polymer, for example polyethylene or PEEK.

Furthermore, the embodiments 80a, 80b, 80c of the cap component according to the invention each have in particular a metallic coating or doping 82a, 82b, 82c, which preferably extends at least partially over the surface of the cap component 80a, 80b, 80c facing the corresponding bone or bone joint part. This coating or doping 82a, 82b, 82c may comprise titanium or calcium, in particular hydroxyapatite.

In addition, each of the embodiments 80a, 80b, 80c shows in particular a pin 83a, 83b, 83c, which preferably serves as a corresponding anti-translation means.

Furthermore, FIG. 8D shows an embodiment 80d of a cap component according to the invention, which is used in particular in conjunction with a femoral condyle 90d. The above explanations can apply analogously to the corresponding base material or a corresponding coating or doping.

It can also be seen that the embodiment 80d has two pins 83d, 84d, which in particular are each round in shape. In particular, these pins 83d, 84d serve not only to prevent the corresponding translation, but also the corresponding rotation.

It should also be mentioned that the sliding surface 89d of the cap component 80d is a convex sliding surface.

However, this can also be flat or planar—as shown in particular in conjunction with a tibia 90e in FIG. 8E. In addition to the flat sliding surface 89e, the embodiment 80e of a cap component according to the invention as shown in FIG. 8E has in particular a base material 81e, as already described above. The coating or doping 82e has also substantially already been described above, which is why there is no need to repeat it.

Furthermore, FIG. 8E shows that the cap component 80e has a total of three pins 83e, 84e, 85e. In particular, these serve not only to prevent the corresponding translation, but also the corresponding rotation.

It is also particularly advantageous if two of the three pins have a round shape, while the third pin has an elliptical or oval shape. For example, the pins 83e, 84e are round, while the pin 85e is elliptical or oval.

Lastly, FIG. 8F depicts an illustration of a further exemplary embodiment of a cap component 80f according to the invention, in particular in conjunction with a tibia-talus joint 90f.

While the tibia 83f of the joint 90f has an insert component 81f in the sense of the invention with a concave sliding surface, the talus 82f of the joint 90f has the cap component according to the invention with a convex sliding surface. While the cap component 80f can be produced using the applicant's convex conventional technology, the insert component 81f is preferably produced using concave titanium plasma spray technology.

In addition, it can be seen from FIG. 8F that the cap component 80f has a pin 84f, which preferably serves a corresponding anti-translation means.

For the sake of completeness, it should also be mentioned that-against the background of FIG. 8E and FIG. 8F or as shown in the example of the tibia—it can be useful to equip a particular bone or a particular bone joint part with a cap component according to the invention in one case and with an insert component according to the invention in another case.

It should also be noted that FIG. 8F additionally illustrates that the invention is particularly suitable for resurfacing the upper ankle joint.

The invention is not limited to the exemplary embodiments discussed above. All features described in the description or features claimed in the claims or features shown in the drawing can be combined with one another in any way within the scope of this invention.

1.-28. (canceled)

29. A cap component for an endoprosthesis, the cap component comprising:
a convex or flat sliding surface;
the cap component being designed to enclose part or all of an associated bone joint part;
the cap component and/or a first base material of the cap component having a modulus of elasticity that is about equal to bone material of the associated bone joint part; and
a facing surface of the cap component facing the associated bone joint part being at least partially coated with a first metal or with particles of the first metal.

30. The cap component of claim 29,
wherein the modulus of elasticity of the cap component and/or the base material of the cap component is less than 10 GPa; and/or
wherein the modulus of elasticity of the sliding surface of the cap component is about equal to the modulus of elasticity of the cap component and/or the first base material of the cap component.

31. The cap component of claim 29,
wherein the first base material of the cap component is softer than a second metal and/or a metal alloy of a second base material of an insert component slidingly cooperating with the cap component,
wherein the second metal includes titanium, iron, chromium, cobalt, or any combination thereof, and/or
wherein the metal alloy includes titanium, iron, chromium, cobalt, or any combination thereof.

32. The cap component of claim 29,
wherein the first metal includes titanium particles, and
wherein the titanium particles are applied using a titanium plasma spray coating process.

33. The cap component of claim 29, wherein the first base material of the cap component includes a plastic.

34. The cap component of claim 29, wherein the cap component has an anti-rotation element arranged on the facing surface of the cap component facing the associated bone joint part.

35. The cap component of claim 34,
wherein the anti-rotation element has the form of a bar, bollard, thread, tooth, cross, star, anchoring pin, or any combination thereof; and/or
wherein the anti-rotation element is arranged circularly and/or at a corresponding pole in relation to the cap component.

36. The cap component of claim 34, wherein the cap component has an anti-translation element arranged on the facing surface of the cap component facing the associated bone joint part.

37. The cap component of claim 36, wherein the anti-translation element has the form of a groove or a cavity.

38. The cap component of claim 36, wherein the anti-rotation element and the anti-translation element are set up to absorb forces and/or moments immediately after implantation and/or for a time after corresponding growth of the bone material of the associated bone joint part.

39. The cap component of claim 29, wherein the cap component has a recess designed to protect zones with critical blood supply during preparation and/or implantation.

40. The cap component of claim 29, wherein the sliding surface of the cap component is spherical on a first side of an equator of the cap component, and the sliding surface of the cap component deviates and/or tapers from being spherical on a second side of the equator of the cap component.

41. The cap component of claim 40,
wherein the sliding surface of the cap component has two different radii, and/or
wherein the sliding surface of the cap component is based on an ellipse or a freely defined curve or line, whereby a femoral head-neck offset of at least 0.5 mm is achieved.

42. The cap component of claim 29, wherein the facing surface of the cap component is spherical on a first side of an equator of the cap component, and the facing surface of the cap component deviates from being spherical on a second side of the equator of the cap component.

43. An endoprosthesis comprising:
an insert component for a first bone joint part of a first bone, the insert component having a sliding surface, and
a cap component for a second bone joint part of a second bone, the cap component including:
a sliding surface;
the cap component being designed to enclose part or all of the second bone joint part;
the cap component and/or a first base material of the cap component having a modulus of elasticity that is about equal to bone material of the second bone joint part; and
a facing surface of the cap component facing the second bone joint part being at least partially coated with a first metal or with particles of the first metal,
wherein the sliding surface of the insert component is in sliding contact with the sliding surface of the cap component.

44. The endoprosthesis according to claim 43,
wherein the insert component is designed to be inserted into the first bone joint part, or to replace the first bone joint part in part or completely, and/or
wherein a facing surface of the insert component facing the first bone joint part is partially or completely coated with the first metal or with particles of the first metal, and/or
wherein a second base material of the insert component includes a second metal or a metal alloy or a metal oxide.

45. The endoprosthesis of claim 43,
wherein the metal oxide includes yttrium oxide, zirconium oxide or aluminum oxide, and/or
wherein the first base material of the cap component is softer than a second metal and/or a metal alloy of the insert component.

46. The endoprosthesis of claim 44,
wherein the second metal of the insert component comprises titanium particles, and
wherein the titanium particles are applied using a titanium plasma spray coating process.

47. The endoprosthesis of claim 43,
wherein the insert component has an anti-rotation and/or anti-translation element, and
wherein the anti-rotation and/or anti-translation element is arranged on a facing surface of the insert component facing the first bone joint part.

48. The endoprosthesis of claim 47, wherein the anti-rotation and/or anti-translation element of the insert component has the form of a tooth or a cavity.
